(19) United States
(12) Patent Application Publication
(10) Pub. No.: US 2022/0219584 A1
(43) Pub. Date: Jul. 14, 2022

(54) ARMREST

(71) Applicant: Faurecia Automotive Seating, LLC, Auburn Hills, MI (US)

(72) Inventors: Cédric KETELS, Auburn Hills, MI (US); Thomas DESSAPT, Auburn Hills, MI (US)

(21) Appl. No.: 17/145,894

(22) Filed: Jan. 11, 2021

(51) Int. Cl. B60N 2/75; B60R 21/02; B60R 7/04; A61L 2/18; A61L 2/10; A61L 9/00

(52) U.S. Cl. CPC B60N 2/79; B60N 2/787; B60R 21/026; B60R 7/043; A61L 2209/16; A61L 2/10; A61L 9/00; A61L 2202/25; A61L 2209/14; A61L 2/18

(57) ABSTRACT

An armrest module includes an armrest base adapted to be coupled to a vehicle and configured to provide a rest area for a passenger's arm and a passenger utility unit coupled to the armrest base. The armrest module further includes a sanitization system coupled to at least one of the armrest base and the passenger utility unit.

### BACKGROUND

The present disclosure relates to armrests in a vehicle, and particularly to armrests that are removable from the vehicle. More particularly, the present disclosure relates to an armrest that is removable from the vehicle and includes sanitization features.

### SUMMARY

According to the present disclosure, a removable armrest module is configured to be coupled to an occupant support in a vehicle. The occupant support includes a seat bottom and a seat back arranged to extend upwardly away from the seat bottom. The seat bottom and the seat back cooperate to define a seat bite region between the seat bottom and the seat back.

In illustrative embodiments, the removable armrest module includes an armrest base adapted to rest on an upper surface of the occupant support, an occupant utility unit coupled to the armrest base, and a built-in sanitization system. The armrest base includes an armrest body that provides a rest area for an occupant's arm and an armrest mount that is configured to be wedged into the seat bite region of the occupant support. The occupant utility unit includes a tray that provides a work surface for the occupant and a privacy wall that separates various compartments in the vehicle for occupant privacy. The sanitization system is configured to disinfect the armrest base, the occupant utility unit, and areas surrounding the armrest module so that the occupant support, the armrest module, and the air in the cabin is sanitized for each occupant using the vehicle.

In illustrative embodiments, the sanitization system may include a surface sanitizer, an air-cleaning system, and a sanitizer dispenser. The surface sanitizer is configured to emit a disinfecting light source to disinfect surfaces of the armrest module, the occupant support, parts of the vehicle, and parts of the occupant. The air-cleaning system is configured to purify and/or sanitize the air in vehicle. The sanitizer dispenser is configured to provide doses of a sanitizing fluid so that the occupant can disinfect their hands and other objects.

Additional features of the present disclosure will become apparent to those skilled in the art upon consideration of illustrative embodiments exemplifying the best mode of carrying out the disclosure as presently perceived.

### BRIEF DESCRIPTION OF THE DRAWINGS

The detailed description particularly refers to the accompanying figures in which:

FIG. 1A is a perspective and diagrammatic view of a plurality of occupant supports within a cabin of a vehicle and showing a removable armrest module, in accordance with the present disclosure, attached to one of the plurality of occupant supports in a rear row of the vehicle and showing that the removable armrest module includes an armrest base, an occupant utility unit including a tray and a privacy wall, and a sanitization system coupled to at least one of the armrest base and the occupant utility unit;

FIG. 1B is an enlarged view of the removable armrest module resting on an upper surface of the rear row occupant supports with a portion of the rear row occupant support cut away to show an armrest mount of the armrest base wedged into a bite line of the rear row occupant supports to retain the removable armrest module to the rear row occupant supports;

FIG. 2 is an overhead view of the armrest module showing that the armrest base is configured to provide a rest area for occupants arms and the privacy wall is configured to divide the rest area into a first sub-region and a second sub-region;

FIG. 3 is a side view of the armrest module with a side panel of the armrest base removed and diagrammatically showing various elements of the sanitization system located within an interior space formed in the armrest base;

FIG. 4 is side view of another embodiment of a surface sanitizer including a plurality of illumination lights and a plurality of disinfecting lights in accordance with the present disclosure; and

FIG. 5 is side view of another embodiment of a surface sanitizer including a plurality of illumination lights and a plurality of disinfecting lights arranged in separate rows in accordance with the present disclosure.

### DETAILED DESCRIPTION

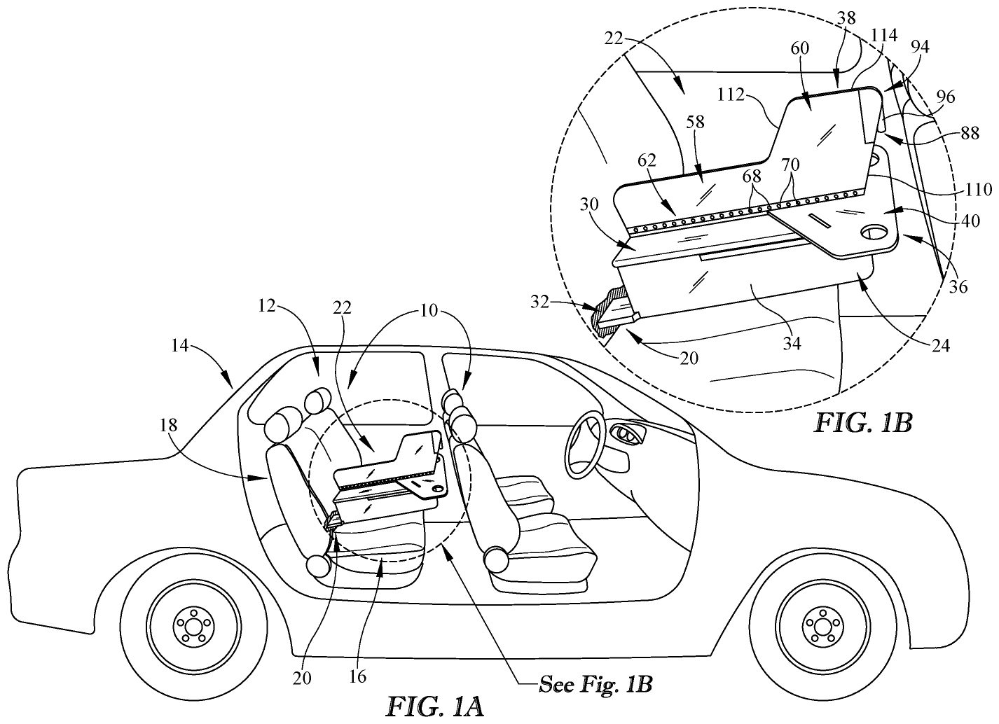

A plurality of occupant supports 10 are arranged within a cabin 12 of a vehicle 14 as shown in FIGS. 1A and 1B. Each occupant support 10 in accordance with the present disclosure includes a seat bottom 16 and a seat back 18 arranged to extend upwardly away from the seat bottom 16. A seat bite region 20 is defined between the seat bottom 16 and the seat back 18 where the seat bottom 16 and the seat back 18 meet. An armrest module 22 may be coupled removably to the occupant support 10 to increase comfort for an occupant seated on occupant support 10 as shown in FIGS. 1A and 1B.

The armrest module 22 includes an armrest base 24, an occupant utility unit 26, and a sanitization system 28 as shown in FIGS. 1A and 1B. The armrest base 24 provides a rest area 30 for one or more arms of occupants seated on the occupant support 10 next to the armrest module 22. The occupant utility unit 26 is coupled to the armrest base 24 and is configured to be used by an occupant to hold items for activities such as eating, drinking, or work related activities. The sanitization system 28 is configured to disinfect the armrest base 24, the occupant utility unit 26, and areas surrounding the armrest module 22 so that the occupant support 10, the armrest module 22, and the air in the cabin 12 is sanitized for each occupant using the vehicle 14.

The armrest base 24 includes an armrest mount 32 and an armrest body 34 as shown in FIGS. 1A and 1B. The armrest mount 32 is configured to mount the armrest module 22 to the occupant support 10 and block the armrest module 22 from undesired movement relative to the occupant support 10. The armrest body 34 positions the rest area 30 in a location that is comfortable for a user of the rest area 30.

The armrest mount 32 may cooperate with portions of the occupant support 10 to mount the armrest module 22 to the occupant support 10 such as by forming wedge that extends into the seat bite region 20. In other embodiments, the armrest mount 32 may use a component of the occupant support 10 such as anchor points located in the seat back 18 or a built-in armrest. In other embodiments the armrest mount 32 may be mounted to one or more components of the vehicle 14 other than occupant support 10, such as a door or a center console between two occupant supports 10.

The armrest body 34 is configured to support the occupant utility unit 26 and the sanitization system 28 above an upper surface of the occupant supports 10 as shown in FIGS. 1A and 1B. The occupant utility unit 26 includes a tray 36 and a privacy wall 38 as shown in FIGS. 1A and 1B. The tray 36 extends horizontally relative to the occupant support 10 and is generally coplanar with the rest area 30 provided by the armrest base 24. The privacy wall 38 extends upwardly from the tray 36 and from the armrest base 24 to provide a barrier between occupants.

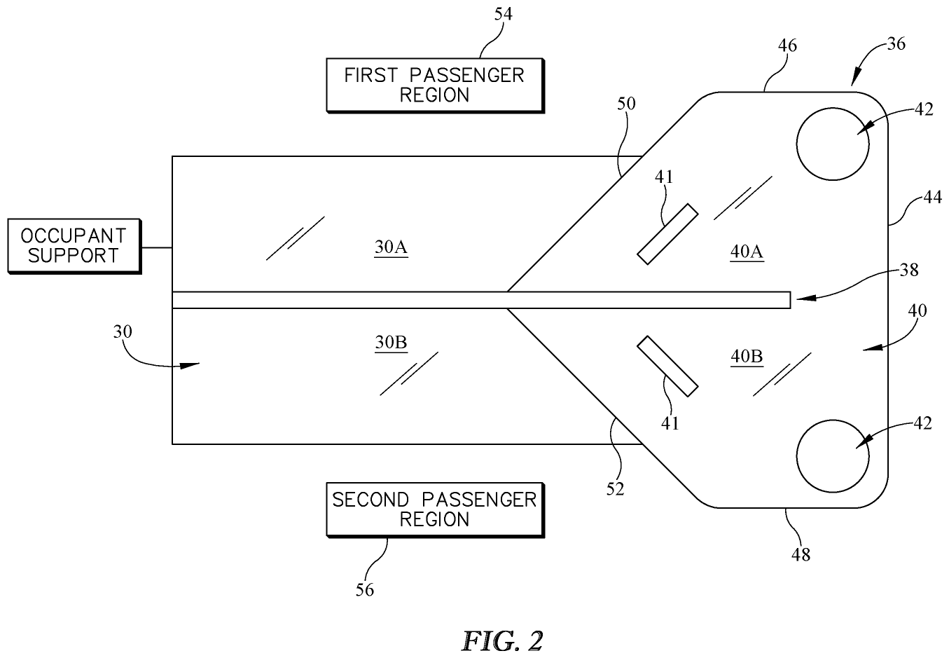

The tray 36 provides a work surface 40 that an occupant may use for activities such as eating, writing, reading, or for holding a device as shown in FIGS. 1A and 1B. The tray 36 may include one or more accessories to hold devices and items such as a mobile-device docking station 41 and/or a cup holder 42. The tray 36 includes a forward edge 44, a pair of parallel side edges 46, 48, and a pair of rearward edges 50, 52 as shown in FIG. 2. The forward edge 44 is arranged forward of a leading end 110 of the privacy wall 38 and is arranged perpendicularly to each side edge 46, 48. The side edges 46, 48 extend rearwardly from the forward edge 44 and are spaced apart from one another on opposite lateral sides of the privacy wall 38. The rearward edges 50, 52 extend rearwardly at an angle relative to each corresponding side edge 46, 48. The rearward edges 50, 52 converge at the privacy wall 38 as they extend rearwardly from each corresponding side edge 46, 48.

The privacy wall 38 divides the rest area 30 and the work surface 40 in half to provide separate sub-regions of the rest area 30 and the work surface 40 on both sides of the privacy wall 38 as shown in FIG. 2. The rest area 30 is divided by the privacy wall 38 into a first rest area 30A and a second rest area 30B. Similarly, the work surface 40 is divided by the privacy wall 38 into a first work surface 40A and a second work surface 40B. The first rest area 30A and the first work surface 40A are located in a first passenger region 54 for use by a first passenger seated in the first passenger region 54. The second rest area 30B and the second work surface 40B are located in a second passenger region 56 for use by a second passenger seated in the second passenger region 56.

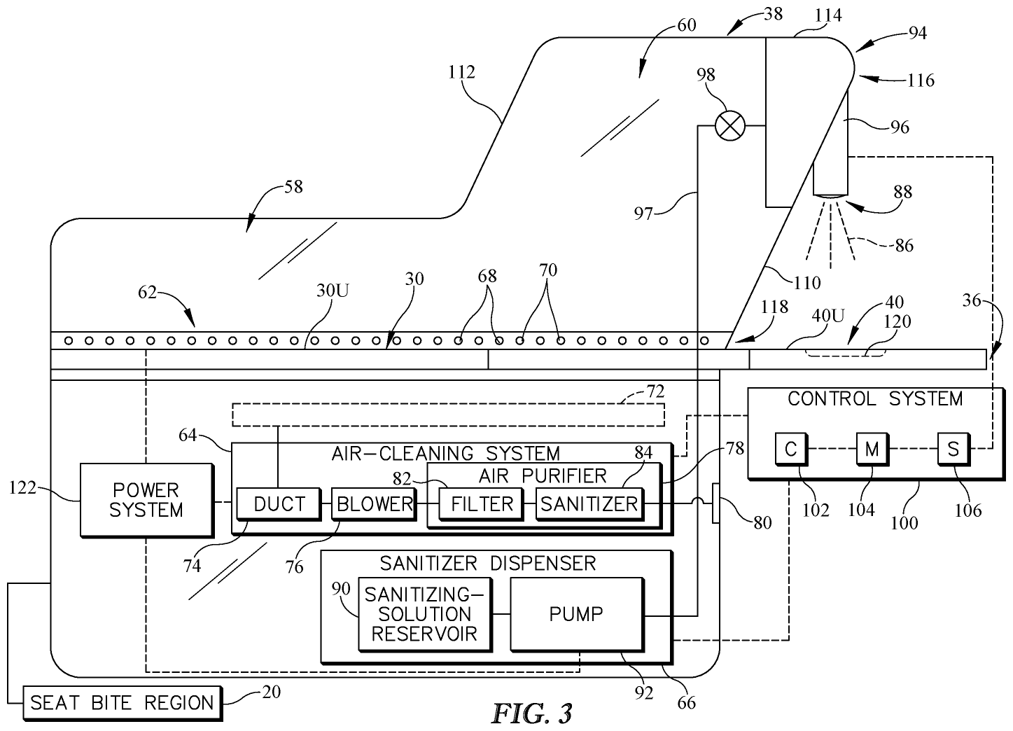

The privacy wall 38 includes a low-elevation, rest-area section 58 and a high-elevation work-surface section 60 as shown in FIGS. 1 and 3. The rest-area section 58 is located rearward of the work-surface section 60. The rest-area section 58 has a height from the armrest base 24 that is lower than a height of the work-surface section 60 relative to the armrest base 24. Passengers seated in both the first passenger region 54 and the second passenger region 56 may be in view of one another over the rest-area section 58 so that they can communicate with one another. At the same time, if one or both of the passengers is using the work surface 40, the work-surface section 60 obscures visibility of any item or device on the tray 36 from the other side of the privacy wall 38 to increase privacy for each passenger. For example, the work surface 40 may include a mobile-device docking station 41 and the work-surface section 60 has a height sufficient to obscure a mobile device (not shown) coupled to the docking station 41. In some embodiments, the privacy wall 38 may have a height sufficient to completely block each passenger's view of the opposite passenger region 54, 56 from the one another.

The sanitization system 28 is configured to reduce or remove contaminants, such as germs, from the rest area 30, the work surface 40, the occupant support 10, components of the vehicle 14 near the armrest module 22, the passengers themselves, and air in the cabin 12. The sanitization system 28 includes a surface sanitizer 62, an air-cleaning system 64, and a sanitizer dispenser system 66 as shown in FIG. 3. The surface sanitizer 62 is coupled to at least one of the armrest base 24 and the privacy wall 38 to disinfect the rest area 30 and the work surface 40. The air-cleaning system 64 is configured to filter and sanitize air through the armrest base 24 to remove contaminants from the air in the cabin 12. The sanitizer dispenser system 66 is configured to dispense a sanitizing fluid 86 from the armrest module 22 so that the passengers can disinfect their hands or items with the sanitizing fluid 86.

The surface sanitizer 62 is illustratively embodied as a sanitizing light strip that is configured to emit a sanitizing light source, such as Ultraviolet-C (UV-C) light, to disinfect surfaces of the armrest module 22 that are frequently contacted by passengers, such as the rest area 30 and the work surface 40. The surface sanitizer 62 is coupled to a lower end of the privacy wall 38 and extends around a perimeter of the lower end of the privacy wall 38 to direct the UV-C light onto an entire upper surface 30U, 40U of the rest area 30 and the work surface 40. The surface sanitizer 62 extends along the lower end of the privacy wall 38 on both sides of the privacy wall 38 so that each of the first and second rest area 30A, 30B and the first and second work surface 40A, 40B is exposed to the sanitizing light source. The sanitizing light source may also disinfect portions of the occupant support 10, other components of the vehicle 14, the passengers, and the air in the cabin 12.

The surface sanitizer 62 may include a plurality of illumination lights 68 that emit an illumination light source and a plurality of disinfecting lights 70 that emit the sanitizing light source as shown in FIG. 3. The illumination lights 68 are configured to illuminate the work surface 40 and portions of each passenger region 54, 56 so that the each passenger can view items and surroundings in dark situations. The disinfecting lights 70 may provide some illumination, but are primarily used to provide the sanitizing light source for the disinfecting purposes previously described. The surface sanitizer 62 may alternate between one illumination light 68 and one disinfecting light 70 as the surface sanitizer 62 extends around the privacy wall 38. It should be noted that all of the lights included in the surface sanitizer 62 may be disinfecting lights 70.

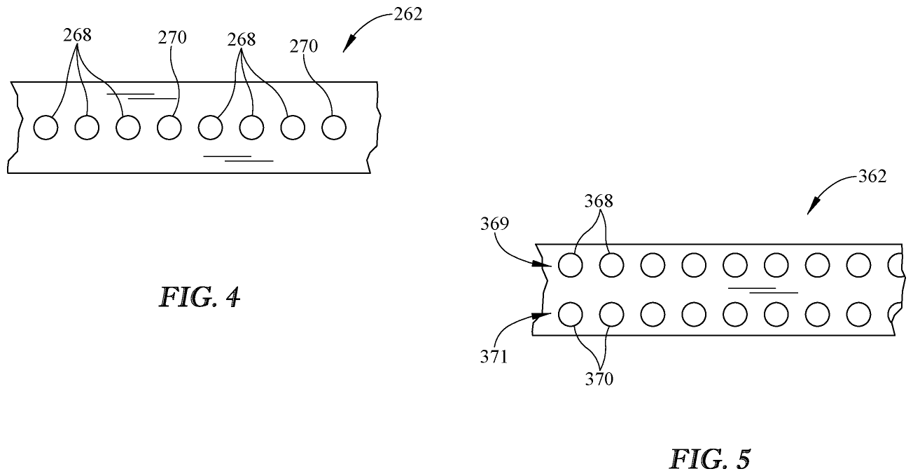

In another embodiment, the armrest module 22 may include a surface sanitizer 262 that is similar to sanitizer 62, but that includes groups of illumination lights 268 that separate each disinfecting light 270 or a group of disinfecting lights 270 as shown in FIG. 4. In yet another embodiment, the armrest module 22 may include a surface sanitizer 362 that is similar to surface sanitizer 62, but that includes a first row of lights 369 including the illumination lights 368 and a second row of lights 371 positioned above or below the first row of lights 369 and including the disinfecting lights 370 as shown in FIG. 5.

The air-cleaning system 64 is configured to discharge sanitized air through an outlet port 72 formed in the armrest base 24 toward a passenger as shown in FIG. 3. The air-cleaning system 64 includes a duct 74, a blower 76 coupled to the duct 74, and an air purifier 78 coupled to the duct 74. The duct 74 extends between the outlet port 72 and an inlet port 80 to transfer air from the inlet port 80 to the outlet port 72. The blower 76 is configured to displace air to flow through the inlet port 80 and the duct 74 to the air purifier 78. The blower 76 provides a sufficient flow rate and pressure to discharge the air through the outlet port 72 so that the passengers are provided with cleaned air when the air-cleaning system 64 is in operation. The air purifier 78 is coupled to the duct between the inlet port 80 and the outlet port 72 to purify and/or sanitize the air flowing through the duct 74 prior to the air being released into the cabin 12 of the vehicle 14 through the outlet port 72.

The air purifier 78 is located in the armrest base 24 and includes a filter 82 and an optional air sanitizer 84 as shown in FIG. 3. The filter 82 is configured to remove or reduce contaminants from the air entering the inlet port 80 to clean the air before it is released back into the cabin 12 of the vehicle 14. The filter 82 may be selected to have a particular minimum efficiency reporting value (MERV) that is tailored to a particular type of contaminant desired to be removed from the air in the cabin 12. High-Efficiency Particulate Air (HEPA) filters or activated carbon filters may also be used. The air sanitizer 84 is configured to sanitize the filter 82 to constantly eradicate any germs that are removed from the air and trapped in the filter 82. The air sanitizer 84 may include a sanitizing light that emits a sanitizing light source, such as a UV-C light source, onto the filter 82.

The sanitizer dispenser system 66 is located in the armrest module 22 and is configured to dispense a sanitizing fluid 86 from a dispenser port 88 so that the passengers can disinfect their hands, other body parts, and items as shown in FIG. 4. The sanitizer dispenser system 66 includes a sanitizer reservoir 90, a pump 92, and a fluid dispenser 94. The sanitizer reservoir 90 is located in the armrest base 24 and stores a supply of the sanitizing fluid 86. The pump 92 is configured to displace doses of the sanitizing fluid 86 from the sanitizer reservoir 90 for discharge through the dispenser port 88. The fluid dispenser 94 discharges the sanitizing fluid 86 and includes a nozzle 96 defining the dispenser port 88, a supply line 97, and a check valve 98. The nozzle 96 may be configured to foam the sanitizing fluid 86 as it exits the dispenser port 88. The check valve 98 is configured to release a single dose of the sanitizing fluid through the dispenser port 88 at a time.

The sanitizer dispenser system 66 may further include a control system 100 that controls discharge of the sanitizing fluid 86 from the dispenser port 88 as shown in FIG. 4. The control system 100 may be coupled to or located in the armrest base 24 and includes a controller 102, a memory storage device 104, and a motion sensor 106. The controller 102 is illustratively embodied as a microprocessor and is configured to receive sensor signals from the motion sensor 106 and output control signals to the pump 92 and the check valve 98 in response to the sensor signals. The memory storage device 104 stores instructions that, when executed by the controller 102, causes the pump to displace a dose of the sanitizing fluid 86 from the sanitizer reservoir 90 and temporarily opens the check valve 98 to discharge a dose of the sanitizing fluid 86 from the dispenser port 88. The motion sensor 106 is configured to sense the presence of a user's hand or an object directly below the nozzle 96 and sends a sensor signal to the controller 102 in response to the sensed presence. Upon receipt of the sensor signal, the controller 102 outputs a command signal to cause the pump to displace a dose of the sanitizing fluid 86 from the sanitizer reservoir 90 and temporarily open the check valve 98 to discharge a dose of the sanitizing fluid 86 from the dispenser port 88. In other embodiments, the pump 92 may be activated manually by a handle or lever to move a dose of the sanitizing fluid 86 from the sanitizer reservoir 90 and out of the dispenser port 88.

The privacy wall 38 has a shape that cooperates with the dispenser system 66 and the tray 36 to improve functionality of the dispenser system 66. When viewed from the side, as shown in FIG. 4, the high-elevation work-surface section 60 of the privacy wall 38 includes a forward end 110, a rear end 112 spaced apart from the forward end 110, and a top end 114 that interconnects upper portions of the forward end 110 and the rear end 112. The forward end 110 and the rear end 112 are generally parallel with one another and extend upwardly and forwardly relative to the tray 36. The dispenser port 88 is coupled to an upper end 116 of the forward end 110 of the privacy wall 38 and is spaced apart from the tray 36. The tray 36 extends forward from a lower edge 118 of the forward end 110 of the privacy wall 38 to locate a portion of the tray directly beneath the dispenser port 88. The tray 36 may be formed to include a depression 120 located directly beneath the dispenser port 88 to collect any excess sanitizing fluid 86 that may drip from the fluid dispenser 94.

The sanitization system 28 further includes a power system 122 that provides an electrical power supply for the surface sanitizer 62, the air-cleaning system 64, and the sanitizer dispenser system 66 as shown in FIG. 3. The power system 122 is an on-board, rechargeable battery in the illustrative embodiment, however, in other embodiments, the power system 122 may be included in the vehicle 14 and coupled to the sanitization system 28 via a suitable electrical connection. Some examples of a suitable electrical connections that can be used include a DC power outlet, an AC power outlet, or another port in the vehicle with electrical charging capabilities such as a USB port, for example. The on-board battery may be connected to one of the electrical connections described above for on-the-go power and/or charging while the sanitization system is in use.

The following numbered clauses include embodiments that are contemplated and non-limiting:

Clause 1. An occupant support comprising a seat bottom, a seat back coupled to the seat bottom, and an armrest module.

Clause 2. The occupant support of clause 1, any other suitable clause, or suitable combination of clauses, wherein the armrest module includes an armrest base coupled to at least one of the seat bottom and the seat back to mount the armrest module in a fixed position relative to the seat bottom and the seat back and to provide a rest area for a passenger's arm.

Clause 3. The occupant support of clause 2, any other suitable clause, or suitable combination of clauses, wherein the armrest module further includes a passenger utility unit coupled to the armrest base.

Clause 4. The occupant support of clause 3, any other suitable clause, or suitable combination of clauses, wherein the passenger utility unit includes a tray coupled to the armrest base to provide a work surface for the passenger.

Clause 5. The occupant support of clause 4, any other suitable clause, or suitable combination of clauses, wherein the passenger utility unit further includes a privacy wall coupled to the armrest base to divide the rest area into a first half configured to be used by a first occupant on a first side of the removable armrest module and a second half configured to be used by a second occupant on an opposite second side of the removable armrest module.

Clause 6. The occupant support of clause 5, any other suitable clause, or suitable combination of clauses, wherein the armrest module further includes a sanitization system coupled to at least one of the armrest base and the passenger utility unit and configured to sanitize at least a portion of the passenger utility unit.

Clause 7. The occupant support of clause 6, any other suitable clause, or suitable combination of clauses, any other suitable clause, or suitable combination of clauses, wherein the sanitization system includes a sanitizing light strip that is coupled to a lower end of the privacy wall and extends around a perimeter of the lower end of the privacy wall to expose the rest area and the work surface to a sanitizing light source emitted by the sanitizing light strip.

Clause 8. The occupant support of clause 6, any other suitable clause, or suitable combination of clauses, wherein the sanitization system further includes an air-cleaning system configured to discharge sanitized air through an outlet port formed in the armrest base toward the passenger.

Clause 9. The occupant support of clause 8, any other suitable clause, or suitable combination of clauses, wherein the air-cleaning system includes a duct that extends through an interior space formed by the armrest base from an inlet port to the outlet port, an air purifier, and a blower configured to displace air through the duct and the air purifier.

Clause 10. The occupant support of clause 9, any other suitable clause, or suitable combination of clauses, wherein the air purifier includes an air filter configured to filter particulates from the air and a sanitizer configured to sanitize the air.

Clause 11. The occupant support of clause 10, any other suitable clause, or suitable combination of clauses, wherein the tray includes a forward edge, a pair of parallel side edges, and a pair of rearward edges, wherein the forward edge is arranged to lie forward of a leading end of the privacy wall and arranged perpendicularly to each side edge, wherein the side edges are arranged to extend rearwardly from the forward edge and being spaced apart from one another on opposite lateral sides of the privacy wall, and wherein the rearward edges extend rearwardly at an angle relative to each corresponding side edge and converge at the privacy wall.

Clause 12. The occupant support of clause 8, any other suitable clause, or suitable combination of clauses, wherein the sanitization system further includes a sanitizer dispenser system configured to dispense a sanitizing fluid from a dispenser port.

Clause 13. The occupant support of clause 12, any other suitable clause, or suitable combination of clauses, wherein the dispenser port is coupled to a forward end of the privacy wall, and the tray extends forward from a lower edge of the privacy wall to locate a portion of the tray directly beneath the dispenser port.

Clause 14. The occupant support of clause 5, any other suitable clause, or suitable combination of clauses, wherein the armrest base includes an armrest housing arranged to lie above the seat bottom to provide the rest area and an armrest mount that extends into a seat bite region of the occupant support defined by a junction between the seat back and the seat bottom where the seat bottom is coupled to the seat back.

Clause 15. An armrest module comprising an armrest base adapted to be coupled to a vehicle and configured to provide a rest area for a passenger's arm.

Clause 16. The armrest of clause 15, any other suitable clause, or suitable combination of clauses, further comprising a passenger utility unit coupled to the armrest base and including a tray coupled to the armrest base to provide a work surface for the passenger.

Clause 17. The armrest of clause 16, any other suitable clause, or suitable combination of clauses, wherein the passenger utility unit further includes a privacy wall coupled to the armrest base to divide the rest area into a first half configured to be used by a first passenger on a first side of the removable armrest module and a second half configured to be used by a second occupant on an opposite second side of the removable armrest module.

Clause 18. The occupant support of clause 17, any other suitable clause, or suitable combination of clauses, further comprising a sanitization system coupled to at least one of the armrest base and the passenger utility unit and configured to sanitize at least a portion of the passenger utility unit and the rest area.

Clause 19. The occupant support of clause 18, any other suitable clause, or suitable combination of clauses, wherein the sanitization system includes a sanitizing light strip that is coupled to a lower end of the privacy wall and extends around a perimeter of the lower end of the privacy wall to expose the rest area and the work surface to a sanitizing light source emitted by the sanitizing light strip.

Clause 20. The occupant support of clause 19, any other suitable clause, or suitable combination of clauses, wherein the sanitization system further includes an air-cleaning system configured to discharge sanitized air through an outlet port formed in the armrest base toward at least one of the first passenger and the second passenger.

Clause 21. The occupant support of clause 20, any other suitable clause, or suitable combination of clauses, wherein the air-cleaning system includes a duct that extends through an interior space formed by the armrest base from an inlet port to the outlet port, an air purifier, and a blower configured to displace air through the duct and the air purifier.

Clause 22. The armrest module of clause 21, any other suitable clause, or suitable combination of clauses, wherein the inlet port is located on a front wall of the armrest base and the armrest base is formed to include a first outlet port on a first lateral side of the armrest base and a second outlet port on an opposite second side of the armrest base.

Clause 23. The occupant support of clause 21, any other suitable clause, or suitable combination of clauses, wherein the air purifier includes an air filter configured to filter particulates from the air and a sanitizer configured to sanitize the air.

Clause 24. The occupant support of clause 20, any other suitable clause, or suitable combination of clauses, wherein the sanitization system further includes sanitizer-dispenser system including a sanitizer dispenser system coupled to one of the privacy wall and the armrest module and configured to dispense a sanitizing fluid from a dispenser port, a sanitizer reservoir located in the armrest module and configured to store the sanitizing fluid, and a pump configured to displace the sanitizing fluid from the sanitizing reservoir to the dispenser port.

Clause 25. The occupant support of clause 24, any other suitable clause, or suitable combination of clauses, wherein the dispenser port is coupled to a forward end of the privacy wall, and the tray extends forward from a lower edge of the privacy wall to locate a portion of the tray directly beneath the dispenser port.

Clause 26. The occupant support of clause 17, any other suitable clause, or suitable combination of clauses, wherein the privacy wall includes a low-elevation, rest-area section and a high-elevation work-surface section, wherein the rest-area section is located rearward of the work-surface section and the rest-area section has a height from the armrest base that is lower than a height of the work-surface section relative to the armrest base.

1. An occupant support comprising
a seat bottom,
a seat back coupled to the seat bottom, and
an armrest module including an armrest base coupled to at least one of the seat bottom and the seat back to mount the armrest module in a fixed position relative to the seat bottom and the seat back and to provide a rest area for a passenger's arm and a passenger utility unit coupled to the armrest base and including a tray coupled to the armrest base to provide a work surface for the passenger and a privacy wall coupled to the armrest base to divide the rest area into a first half configured to be used by a first occupant on a first side of the removable armrest module and a second half configured to be used by a second occupant on an opposite second side of the removable armrest module.

2. The occupant support of claim 1, wherein the armrest module further includes a sanitization system coupled to at least one of the armrest base and the passenger utility unit and configured to sanitize at least a portion of the passenger utility unit.

3. The occupant support of claim 2, wherein the sanitization system includes a sanitizing light strip that is coupled to a lower end of the privacy wall and extends around a perimeter of the lower end of the privacy wall to expose the rest area and the work surface to a sanitizing light source emitted by the sanitizing light strip.

4. The occupant support of claim 2, wherein the sanitization system further includes an air-cleaning system configured to discharge sanitized air through an outlet port formed in the armrest base toward the passenger.

5. The occupant support of claim 4, wherein the air-cleaning system includes a duct that extends through an interior space formed by the armrest base from an inlet port to the outlet port, an air purifier, and a blower configured to displace air through the duct and the air purifier.

6. The occupant support of claim 5, wherein the air purifier includes an air filter configured to filter particulates from the air and a sanitizer configured to sanitize the air.

7. The occupant support of claim 6, wherein the tray includes a forward edge, a pair of parallel side edges, and a pair of rearward edges, wherein the forward edge is arranged to lie forward of a leading end of the privacy wall and arranged perpendicularly to each side edge, wherein the side edges are arranged to extend rearwardly from the forward edge and being spaced apart from one another on opposite lateral sides of the privacy wall, and wherein the rearward edges extend rearwardly at an angle relative to each corresponding side edge and converge at the privacy wall.

8. The occupant support of claim 4, wherein the sanitization system further includes a sanitizer dispenser system configured to dispense a sanitizing fluid from a dispenser port.

9. The occupant support of claim 8, wherein the dispenser port is coupled to a forward end of the privacy wall, and the tray extends forward from a lower edge of the privacy wall to locate a portion of the tray directly beneath the dispenser port.

10. The occupant support of claim 1, wherein the armrest base includes an armrest housing arranged to lie above the seat bottom to provide the rest area and an armrest mount that extends into a seat bite region of the occupant support defined by a junction between the seat back and the seat bottom where the seat bottom is coupled to the seat back.

11. An armrest module comprising
an armrest base adapted to be coupled to a vehicle and configured to provide a rest area for a passenger's arm, and
a passenger utility unit coupled to the armrest base and including a tray coupled to the armrest base to provide a work surface for the passenger and a privacy wall coupled to the armrest base to divide the rest area into a first half configured to be used by a first passenger on a first side of the removable armrest module and a second half configured to be used by a second occupant on an opposite second side of the removable armrest module.

12. The occupant support of claim 11, further comprising a sanitization system coupled to at least one of the armrest base and the passenger utility unit and configured to sanitize at least a portion of the passenger utility unit and the rest area.

13. The occupant support of claim 12, wherein the sanitization system includes a sanitizing light strip that is coupled to a lower end of the privacy wall and extends around a perimeter of the lower end of the privacy wall to expose the rest area and the work surface to a sanitizing light source emitted by the sanitizing light strip.

14. The occupant support of claim 13, wherein the sanitization system further includes an air-cleaning system configured to discharge sanitized air through an outlet port formed in the armrest base toward at least one of the first passenger and the second passenger.

15. The occupant support of claim 14, wherein the air-cleaning system includes a duct that extends through an interior space formed by the armrest base from an inlet port to the outlet port, an air purifier, and a blower configured to displace air through the duct and the air purifier.

16. The armrest module of claim 15, wherein the inlet port is located on a front wall of the armrest base and the armrest base is formed to include a first outlet port on a first lateral side of the armrest base and a second outlet port on an opposite second side of the armrest base.

17. The occupant support of claim 15, wherein the air purifier includes an air filter configured to filter particulates from the air and a sanitizer configured to sanitize the air.

18. The occupant support of claim 14, wherein the sanitization system further includes sanitizer-dispenser system including a sanitizer dispenser system coupled to one of the privacy wall and the armrest module and configured to dispense a sanitizing fluid from a dispenser port, a sanitizer reservoir located in the armrest module and configured to store the sanitizing fluid, and a pump configured to displace the sanitizing fluid from the sanitizing reservoir to the dispenser port.

19. The occupant support of claim 18, wherein the dispenser port is coupled to a forward end of the privacy wall, and the tray extends forward from a lower edge of the privacy wall to locate a portion of the tray directly beneath the dispenser port.

20. The occupant support of claim 11, wherein the privacy wall includes a low-elevation, rest-area section and a high-elevation work-surface section, wherein the rest-area section is located rearward of the work-surface section and the rest-area section has a height from the armrest base that is lower than a height of the work-surface section relative to the armrest base.
